(19) United States
(12) Patent Application Publication
(10) Pub. No.: US 2013/0109956 A1
(43) Pub. Date: May 2, 2013

(54) PREMATURE NEONATE LIFE SUPPORT ENVIRONMENTAL CHAMBER FOR USE IN MRI/NMR DEVICES

(71) Applicant: Uri Rapoport, Moshav Ben Shemen (IL)

(72) Inventor: Uri Rapoport, Moshav Ben Shemen (IL)

(73) Assignee: ASPECT IMAGING LTD., Shoham (IL)

(21) Appl. No.: 13/808,476

(22) PCT Filed: Jul. 7, 2011
(86) PCT No.: PCT/IL11/00537

§ 371 Date: Jan. 11, 2013

Related U.S. Application Data
(60) Provisional application No. 61/361,936, filed on Jul. 7, 2010.

(51) Int. Cl. A61G 11/00; A61B 5/055

(52) U.S. Cl. CPC A61G 11/005; A61B 5/055
USPC 600/411; 600/22

(57) ABSTRACT

The present invention premature neonate life support environmental cradle (SEC), adapted by means of shape and size to be accommodated within (i) an incubator for premature neonates; and (ii) a measurement device, especially an MRI device. The SEC is configured for a first at least one open position for accommodating said neonate whilst the SEC is within the incubator, and a second closed position for confinement of the neonate within a sealed volume of incubator atmosphere within the SEC such that the integrity of the confinement is maintained upon removal of the closed SEC containing the neonate from the incubator.

### FIELD OF THE INVENTION

The present invention relates to equipment for MRI imaging of premature neonates. In particular, it pertains to means and methods for enabling MRI examinations and measurements to be taken of premature neonates without detaching the neonates from life-support systems.

### BACKGROUND OF THE INVENTION

A well-known technical problem in the design of an MRI/NMR device is that of the fringing magnetic fields. These fringing magnetic fields can cause interference with nearby devices and lead to safety concerns arising from the possibility of placing magnetic material in propinquity to the MRI/NMR device as well as limiting the proximity of two MRI/NMR devices or other devices. This limitation results in the fact that almost invariably the MRI/NMR device is stored in a special isolated facility. This last problem is of particular concern for cases in which it is required to take MRI imaging examinations and measurements of a premature neonate without transferring the premature neonate between a premature neonate intensive-care ward and an MRI imaging facility, without decoupling and disconnecting the premature neonate from life-support systems.

The following prior art are believed to be relevant to the current status of the art:

US Published Patent Application No. 2007/0232894 to Feenan describes an extendable carriage configured to shuttle between the neonate incubator and the magnetic resonance incubator system.

U.S. Pat. No. 6,611,702 to Rohling et al. describes an enclosure within the MRI device and adapted to maintain a selected environment with couplings to life-support mechanisms. An RF coil is located within the enclosure.

The website currently available at http://www.sciencedaily.com/videos/2005/1211-inside_the_preemie_brain.htm shows an incubator in use with an MRI device in which the premature neonate is transferred to a transportable incubator for examination.

The above-cited references, describe incubator devices in which the patient is removed from an incubator located in a hospital ward and placed within an MRI-incubator prior to the MRI examinations.

U.S. Pat. Nos. 5,800,335 and 7,278,962 to Koch and Lonneker-Lammers, respectively, describe a specialized incubator for use in tomography. These references do not relate to the problem of transferring a neonate from a ward-incubator to an MRI-compatible incubator.

The above-cited references do not provide a system or method enabling a premature neonate to undergo an MRI/NMR examination e.g., in the premature neonate intensive-care ward and require that the premature neonate be detached from the life-supporting systems during transfer to the MRI-compatible incubator. Thus, during placing the patient, such as a neonate, neonate in the prior art incubator, the patient or neonate is detached from the life-support systems, which can endanger the health of the patient.

Therefore, there is a long felt and hitherto unmet need for providing improved incubator devices.

### SUMMARY OF THE INVENTION

One object of the invention is to disclose a premature neonate life support environmental cradle (SEC), adapted by means of shape and size to be accommodated within (i) an incubator for premature neonates; and (ii) a measurement device; the SEC is configured for a first at least one open position for accommodating the neonate whilst the SEC is within the incubator, and a second closed position for confinement of the neonate within a sealed volume of incubator atmosphere within the SEC such that the integrity of the confinement is maintained upon removal of the closed SEC containing the neonate from the incubator.

Another object of the invention is to disclose an MRI-dedicated premature neonate life support environmental cradle (SEC) as defined above, wherein the MRI-dedicated SEC is constructed from materials permeable to magnetic fields; and wherein the measurement device is an MRI device.

Another object of the invention is to disclose a SEC as defined above, wherein the SEC further comprising an adaptor for connection to a closed life support system for the neonate.

Another object of the invention is to disclose a SEC as defined above, wherein the SEC further comprising one or more hand access ports and at least two flexible non-resilient lightweight sealing gas-tight flaps (SFs), disposed within the same plane of the port aperture in an overlapping manner, entirely enclosing the port aperture for handling the neonate.

Another object of the invention is to disclose a SEC as defined above, wherein the SEC wherein the SEC is characterized by an at least partially reversibly collapsible or deployable structure.

Another object of the invention is to disclose a SEC as defined above, wherein the at least partially reversibly collapsible structure is constructed from a plurality of curved or polygonal flaps or sections adapted by means of size and shape to construct a continuous envelope having an open bore accommodating the neonate.

Another object of the invention is to disclose premature neonate life support incubator (SEI) adapted by means of shape and size to be accommodated within a measurement device; the SEI is made of materials that do not interfere with the measurement; the SEI is configured for a closed position for confinement of the neonate within a sealed volume of the SEI such that the integrity of the confinement is maintained upon transportation between the SEI containing the neonate and the measurement device.

Another object of the invention is to disclose an MRI-dedicated premature neonate life support incubator (MRI-SEI) adapted by means of shape and size to be accommodated within an MRI device; the MRI-SEI is made of magnetically permeable materials; the MRI-SEI is configured for a closed position for confinement of the neonate within a sealed volume of the MRI-SEI such that the integrity of the confinement is maintained upon both (i) transportation of the MRI-SEI containing the neonate to the MRI device and back, and (ii) during the measurement.

Another object of the invention is to disclose a method of taking measurements of a premature neonate without exposing the same to an environment outside its incubator; the method comprising steps of obtaining a premature neonate life support environmental cradle (SEC), providing the SEC by means of shape and size to be accommodated within (i) an incubator for premature neonates; and (ii) a measurement device; placing the SEC within the incubator; opening the SEC within the incubator; accommodating the neonate within the SEC in its at least one open position; before carrying out the measurements, closing the SEC within the incubator thereby confining within the SEC (i) a volume of incubator atmosphere and (ii) the neonate; exiting the SEC in its closed position from the incubator; and transferring the SEC containing the neonate to the measurement device and carrying out the measurements; without exposing the neonate to an external environment; wherein the step of transferring the neonate within the confined SEC is executed without exposing the neonate to an external environment.

Another object of the invention is to disclose the method defined above, wherein the method is especially adapted for taking MRI measurements of a premature neonate without exposing the same to an environment outside its incubator; wherein the step of obtaining a the SEC includes a step of constructing the SEC from materials permeable to magnetic fields; and the step of containing the neonate to the measurement device includes selecting the measurement device to be an MRI device.

Another object of the invention is to disclose a method of taking measurements of a premature neonate without exposing the same to an environment outside his incubator; the method comprising steps of obtaining a premature neonate life support incubator (SEI) adapted by means of shape and size to be accommodated within a measurement device; and constructing the SEI with materials that do not interfere with the measurement; configuring the SEI for a closed position for confinement of the neonate within a sealed volume of the SEI; accommodating the neonate within the SEI; before carrying out the measurements, transferring the SEI containing the neonate to the measurement device and carrying out the measurements; without exposing the neonate to an external environment. Thus, the integrity of the confinement is maintained upon both transportation of the SEI containing the neonate to the measurement device and back and along the measurement

Another object of the invention is to disclose the method as defined above, especially adapted for taking MRI measurements of a premature neonate without exposing the same to an environment outside his incubator; wherein the step of obtaining a the SEC including a step of constructing the SEC from materials permeable to magnetic fields; and the step of carrying out the measurements including selecting the measurement device to be an MRI device.

Another object of the invention is to disclose a method of life support of a premature neonate during MRD measurements; the method comprising the steps of providing a neonate incubator permeable to magnetic fields; providing a premature support environmental chamber configured to contain the neonate incubator and having at least one of the life support services conventionally supplied to same, such that the neonate incubator is continuously provided with same; placing premature neonate into the incubator; and operating at least one life support service to the premature neonate; transferring the incubator containing the premature neonate into the SEC; wherein the step of transferring is made without decoupling the at least one life support service and further wherein the SEC operates from within a portable MRD device.

Another object of the invention is to disclose a premature neonate life-support environmental chamber (SEC) for MRI measurements comprising an MRI-compatible life-saving cubicle for supplying at least one life-support service to a premature neonate having an adaptable geometrical shape for insertion into an MRI device and comprising at least one magnetically permeable material, and a life-support coupling system for coupling the MRI-compatible life-saving cubicle to the at least one life-support system having an adaptable geometrical shape for insertion into the MRI device and comprising: a coupling/decoupling mechanism, and at least one magnetically permeable material, wherein the MRI-compatible life-saving cubicle remains coupled to the life-support system during placing the premature neonate in the MRI-compatible life-saving cubicle.

Another object of the invention is to disclose the device as defined above, wherein the at least one life-support service is selected from the group consisting of air, water, gas and fluid exchange, drug delivery, perfusions, waste removal, transfusions, anesthetic gas and any combination thereof.

Another object of the invention is to disclose a method for performing MRI measurements in a premature neonate life-support environmental chamber (SEC) comprising: providing an MRI device for performing the MRI measurements on a premature neonate; placing the premature neonate in an MRI-compatible life-saving cubicle; coupling the MRI-compatible cubicle to at least one life-support system; inserting the coupled-MRI-compatible life-saving cubicle into the MRI device, performing MRI measurements on the premature neonate, wherein concomitantly with the inserting the coupled-MRI-compatible cubicle into the MRI device, the life-support system remains coupled to the MRI-compatible cubicle providing the premature neonate with continuous at least one life-support service.

Another object of the invention is to disclose the method as defined above, wherein the MRI-compatible life-saving cubicle comprises magnetically permeable materials.

A method of supplying at least life-support service to a premature neonate during MRI imaging measurements comprising: providing a premature neonate environmental chamber (SEC) comprising an MRD device for MRI imaging of the premature neonate, and a premature neonate incubator having magnetically permeable walls; placing the premature neonate in the incubator; coupling the incubator to at least one of the life-support system for providing the at least life-support service to the premature neonate during the MRD imaging the premature neonate; operating at least one life-support service to the premature neonate; inserting the incubator accommodating the premature neonate into the SEC; wherein the inserting is performed while the incubator remains coupled to the at least one life-support system.

Magnetically permeable materials include, inter alia, materials, such as a polymethacrylate substance, such as polymethyl methacrylate (PMMA), Plexiglas and Perspex.

The life-support systems typically provide the following environmental properties to the neonate while placed in the incubator of the present invention, such as oxygen, air temperature and humidity, gas and fluid exchange, drug delivery, perfusions, waste removal, transfusions, anesthetic gas and any other necessary fluids and substances.

### BRIEF DESCRIPTION OF THE DRAWINGS

In order to understand the invention and to see how it may be implemented in practice, a few preferred embodiments will now be described, by way of non-limiting example only, with reference to the accompanying drawings, in which:

FIGS. 1 and 3 present an out-of-scale illustration (perspective view) of a SEC according to some embodiments of the invention;

FIGS. 3a and 3b present a prior art two-step procedure;

FIG. 4 presents an out-of-scale a one-step procedure according to one embodiment of the invention; and

FIG. 5 presents an out-of-scale another one-step procedure and a SEI device according to one embodiment of the invention.

### DETAILED DESCRIPTION OF A PREFERRED EMBODIMENT OF THE PRESENT INVENTION

The following description is provided, alongside all chapters of the present invention, so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a premature neonate life support environmental chamber and methods using the same.

The present invention provides a premature neonate life Support Environmental Chamber (hereinafter ‘SEC’), permeable to magnetic fields for use in a portable MRI/NMR device, such as an MRD device. This environmental chamber SEC is adapted to accommodate any neonate incubator permeable to magnetic fields. Hence, the SEC is configured such that the SEC and the incubator include a closed life support system for the neonate.

The term ‘anesthetic gas’ refers hereinafter to any gas selected from a group consisting of Nitrous oxide (N₂O), Halothane, Enflurane, Isoflurane, Sevoflurane, Desflurane and Xenon, water, oxygen or any combination thereof.

The term ‘magnetic resonance device’ (MRD) applies hereinafter to any Magnetic Resonance Imaging (MRI) device, any Nuclear Magnetic Resonance (NMR) spectroscope, any Electron Spin Resonance (ESR) spectroscope, any Nuclear Quadruple Resonance (NQR) or any combination thereof. It is herein acknowledged that embodiments of the present invention are adapted for use with any combination of the aforementioned MRD devices.

As used herein, the term “neonate” refers to any object or living creature inserted in whole or in part within an incubator.

As used herein, the term “plurality” refers in a non-limiting manner to any integer equal or greater than 1.

The term ‘about’ applies hereinafter to a measure being ±25% of the defined value.

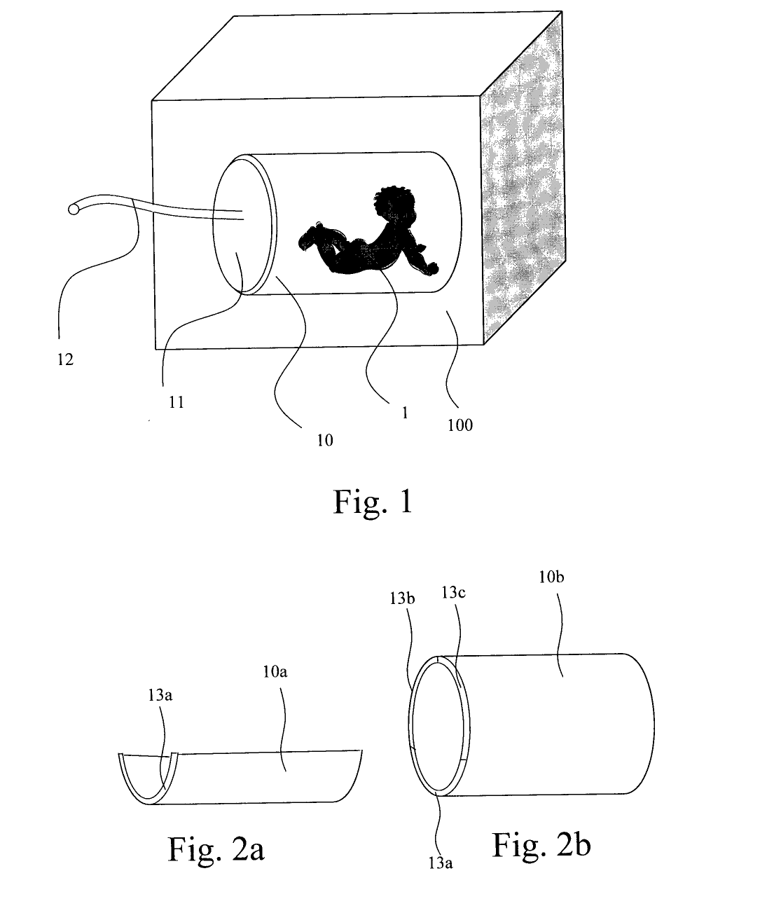

Reference is now made to FIG. 1 which discloses in an illustrative manner a premature neonate life support environmental cradle (SEC, 10), adapted by means of shape and size to be accommodated within (i) an incubator (100) for premature neonates (1); and (ii) a measurement device (not shown). The SEC, according to one embodiment of the invention, further comprises an adaptor (11) for connection to a closed life support system (12) for the neonate. The support is one or more member of a group consisting of anesthetic gas, air conditioning, water, food supply, medicines, infusions, electronics, cables to various medical devices etc.

Reference is now made to FIGS. 2a and 2b which illustrates a SEC which is configured for a first at least one open position (FIG. 2a. 10a) for accommodating the neonate whilst the SEC is within the incubator, and a second closed position (FIG. 2b. 10b) for confinement of the neonate within a sealed volume of incubator atmosphere within the SEC such that the integrity of the confinement is maintained upon removal of the closed SEC containing the neonate from the incubator. This confinement may be constructed to be either fluid tight, air tight etc.

According to one embodiment of the invention, this at least partially reversibly collapsible or deployable structure (10a-10b) is constructed from a plurality of curved or polygonal flaps or sections adapted by means of size and shape to form a continuous envelope having an open bore accommodating the neonate. Here, one flap (13a) forms the cradle and two other flaps (13b and 13c) form the walls.

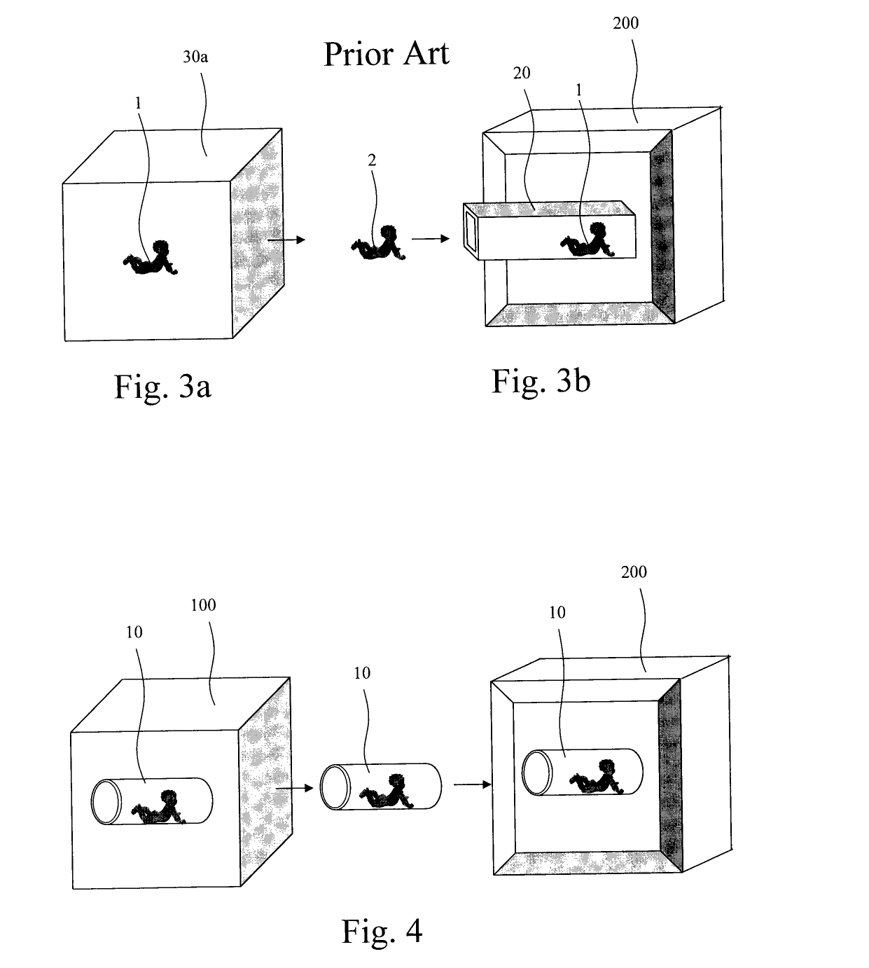

Reference is now made to FIGS. 3a-3b, which present the prior art: a neonate (1) is accommodated within an incubator (10b). Before taking the measurement, e.g., an MRI measurement by means of an MRI (200) positioned in a remote location, the neonate is evacuated from its incubator, and may even be separated from the support system for a short period of time, and then the neonate positioned within an MRI device. In some cases, a specially designed MRI-incubator (20) is used, to support and fix the neonate while taking the measurement. After the measurement is ended, the neonate is evacuated from the measurement device, and again, exposed to the ambient dangerous environment before returning to his incubator. This two-step procedure is thus dangerous for the neonate.

Reference is now made to FIG. 4 which demonstrate the single-step procedure of the present invention, in which medical examinations and measurements are performed using a measurement device without detaching the neonate from the life-support systems, which would endanger his life: The neonate is regularly positioned in an open position SEC within an incubator 100. Before the measurement, the SEC is configured in its closed position (10). Now the neonate, still in connection to the life support systems (not shown), is safely transported within the SEC 10 to a measurement device (200), here an MRI device. SEC 10 is made of materials permeable to magnetism, thus the neonate is fixed and supported in his safe environment. The return of the neonate to its incubator is similarly easy and safe.

FIG. 4 also schematically presents a method of taking measurements of a premature neonate without exposing the same to an environment outside its incubator. This method comprises steps of obtaining a premature neonate life support environmental cradle (SEC, 10), providing the SEC by means of shape and size to be accommodated within (i) an incubator (100) for premature neonates; and (ii) a measurement device (200); placing SEC 10 within incubator 100; opening SEC 10 within incubator 100; accommodating a neonate within the SEC in its at least one open position; before carrying out the measurements, closing SEC 10 within the incubator thereby confining within SEC (i) a volume of incubator 10 atmosphere and (ii) the neonate; exiting SEC 10 in its closed position from incubator 100; and transferring SEC 10 containing the neonate to measurement device (e.g., an MRI device 200) and carrying out the measurements without exposing the neonate to an external environment.

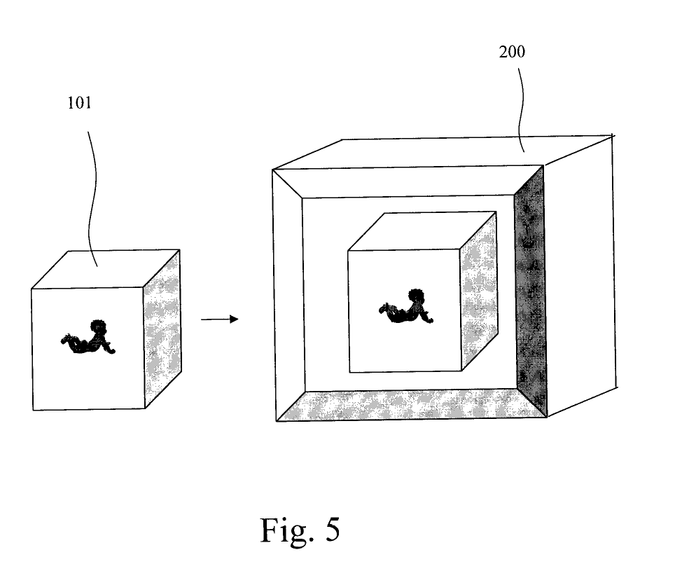

Reference is now made to FIG. 5 which illustrates an MRI-dedicated premature neonate life support incubator (MRI-SEI, 101) according to yet another embodiment of the invention. MRI-SEI (101) is made of magnetically permeable materials. The MRI-SEI is configured for a closed position for confinement of a neonate within a sealed volume of the MRI-SEI such that the integrity of the confinement is maintained upon both (i) transportation of the MRI-SEI containing the neonate to the MRI device (200) and back, and (ii) during the measurement.

FIG. 5 also schematically presents a method of taking measurements of a premature neonate without exposing the same to an environment outside its incubator. The method comprises steps of obtaining the premature neonate life support incubator (SEI, here also 101) adapted by means of shape and size to be accommodated within a measurement device (here also 200); and constructing the SEI with materials that do not interfere with the measurement; configuring the SEI for a closed position for confinement of the neonate within a sealed volume of the SEI; accommodating the neonate within the SEI; before carrying out the measurements, transferring the SEI containing the neonate to the measurement device and carrying out the measurements; without exposing the neonate to an external environment; such that the integrity of the confinement is maintained upon both transportation of the SEI containing the neonate to the measurement device and back and along the measurement

1. A premature neonate life support environmental cradle (SEC), adapted by means of shape and size to be accommodated within (i) an incubator for premature neonates; and (ii) a measurement device; said SEC is configured for a first at least one open position for accommodating said neonate whilst said SEC is within said incubator, and a second closed position for confinement of said neonate within a sealed volume of incubator atmosphere within said SEC such that the integrity of said confinement is maintained upon removal of said closed SEC containing said neonate from said incubator.

2. An MRI-dedicated premature neonate life support environmental cradle (SEC) according to claim 1, wherein said MRI-dedicated SEC is constructed from materials permeable to magnetic fields; and wherein said measurement device is an MRI device.

3. The SEC according to claim 1, further comprising an adaptor for connection to a closed life support system for said neonate.

4. The SEC according to claim 1, further comprising one or more hand access ports and at least two flexible non-resilient lightweight sealing gas-tight flaps, disposed within the same plane of the port aperture in an overlapping manner, entirely enclosing the port aperture for handling said neonate.

5. The SEC according to claim 1 wherein said SEC is characterized by an at least partially reversibly collapsible or deployable structure.

6. The SEC according to claim 5 wherein said at least partially reversibly collapsible structure is constructed from a plurality of curved or polygonal flaps or sections adapted by means of size and shape to construct a continuous envelope having an open bore accommodating said neonate.

7. A premature neonate life support incubator (SEI) adapted by means of shape and size to be accommodated within a measurement device; said SEI is made of materials that do not interfere with said measurement; said SEI is configured for a closed position for confinement of said neonate within a sealed volume of said SEI such that the integrity of said confinement is maintained upon transportation between said SEI containing said neonate and the measurement device.

8. An MRI-dedicated premature neonate life support incubator (MRI-SEI) adapted by means of shape and size to be accommodated within an MRI device; said MRI-SEI is made of magnetically permeable materials; said MRI-SEI is configured for a closed position for confinement of said neonate within a sealed volume of said MRI-SEI such that the integrity of said confinement is maintained upon both (i) transportation of said MRI-SEI containing said neonate to the MRI device and back, and (ii) during said measurement.

9. A method of taking measurements of a premature neonate without exposing the same to an environment outside its incubator; said method comprising steps of
a. obtaining a premature neonate life support environmental cradle (SEC), providing said SEC by means of shape and size to be accommodated within (i) an incubator for premature neonates; and (ii) a measurement device;
b. placing said SEC within said incubator;
c. opening said SEC within said incubator;
d. accommodating said neonate within said SEC in its at least one open position;
e. before carrying out said measurements, closing said SEC within said incubator thereby confining within said SEC (i) a volume of incubator atmosphere and (ii) said neonate;
f. exiting said SEC in its closed position from said incubator; and
g. transferring said SEC containing said neonate to said measurement device and carrying out said measurements; without exposing said neonate to an external environment;
wherein said step of transferring said neonate within said confined SEC is executed without exposing said neonate to an external environment.

10. The method of claim 8, especially adapted for taking MRI measurements of a premature neonate without exposing the same to an environment outside its incubator; wherein said step of obtaining a said SEC includes a step of constructing said SEC from materials permeable to magnetic fields; and said step of containing said neonate to said measurement device includes selecting said measurement device to be an MRI device.

11. A method of taking measurements of a premature neonate without exposing the same to an environment outside his incubator; said method comprising steps of
a. obtaining a premature neonate life support incubator (SEI) adapted by means of shape and size to be accommodated within a measurement device; and constructing said SEI with materials that do not interfere with said measurement; configuring said SEI for a closed position for confinement of said neonate within a sealed volume of said SEI;
b. accommodating said neonate within said SEI; and
c. before carrying out said measurements, transferring said SEI containing said neonate to said measurement device and carrying out said measurements; without exposing said neonate to an external environment;
such that the integrity of said confinement is maintained upon both transportation of said SEI containing said neonate to the measurement device and back and along said measurement

12. The method of claim 11, especially adapted for taking MRI measurements of a premature neonate without exposing the same to an environment outside his incubator;
wherein said step of obtaining a said SEC including a step of constructing said SEC from materials permeable to magnetic fields; and said step of carrying out said measurements including selecting said measurement device to be an MRI device.

13. A method of life support of a premature neonate during MRD measurements; said method comprising the steps of
a. providing a neonate incubator permeable to magnetic fields;
b. providing a premature support environmental chamber configured to contain said neonate incubator and having at least one of the life support services conventionally supplied to same, such that said neonate incubator is continuously provided with same;
c. placing premature neonate into said incubator; and
d. operating at least one life support service to said premature neonate;
e. transferring said incubator containing said premature neonate into said SEC
wherein said step of transferring is made without decoupling said at least one life support service and further wherein said SEC operates from within a portable MRD device.

14. A premature neonate life-support environmental chamber (SEC) for MRI measurements comprising
a. an MRI-compatible life-saving cubicle for supplying at least one life-support service to a premature neonate having an adaptable geometrical shape for insertion into an MRI device and comprising at least one magnetically permeable material, and
b. a life-support coupling system for coupling said MRI-compatible life-saving cubicle to said at least one life-support system having an adaptable geometrical shape for insertion into said MRI device and comprising:
i. a coupling/decoupling mechanism, and
ii. at least one magnetically permeable material,
wherein said MRI-compatible life-saving cubicle remains coupled to said life-support system during placing said premature neonate in said MRI-compatible life-saving cubicle.

15. The SEC according to claim 14, wherein said at least one life-support service is selected from the group consisting of air, water, gas and fluid exchange, drug delivery, perfusions, waste removal, transfusions, anesthetic gas and any combination thereof.

16. A method for performing MRI measurements in a premature neonate life-support environmental chamber (SEC) comprising:
a. providing an MRI device for performing said MRI measurements on a premature neonate;
b. placing said premature neonate in an MRI-compatible life-saving cubicle;
c. coupling said MRI-compatible cubicle to at least one life-support system;
d. inserting said coupled-MRI-compatible life-saving cubicle into said MRI device,
e. performing MRI measurements on said premature neonate,
wherein concomitantly with said inserting said coupled-MRI-compatible cubicle into said MRI device, said life-support system remains coupled to said MRI-compatible cubicle providing said premature neonate with continuous at least one life-support service.

17. The method according to claim 16, wherein said MRI-compatible life-saving cubicle comprises magnetically permeable materials.

18. A method of supplying at least life-support service to a premature neonate during MRI imaging measurements comprising:
a. providing a premature neonate environmental chamber (SEC) comprising
i. an MRD device for MRI imaging of said premature neonate, and
ii. a premature neonate incubator having magnetically permeable walls;
b. placing said premature neonate in said incubator;
c. coupling said incubator to at least one of the life-support system for providing said at least life-support service to said premature neonate during said MRD imaging said premature neonate.
d. operating at least one life-support service to said premature neonate;
e. inserting said incubator accommodating said premature neonate into said SEC
wherein said inserting is performed while said incubator remains coupled to said at least one life-support system.
